(19) United States
(12) Patent Application Publication
(10) Pub. No.: US 2009/0060878 A1
(43) Pub. Date: Mar. 5, 2009

(54) Oral Compositions, Products And Methods Of Use

(75) Inventors: Jeffrey Warren Clymer, Mason, OH (US); Paul John Rennie, Surrey (GB)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company, Cincinnati, OH (US)

(21) Appl. No.: 12/201,087

(22) Filed: Aug. 29, 2008

Related U.S. Application Data
(60) Provisional application No. 60/967,412, filed on Sep. 4, 2007.

(51) Int. Cl. A61K 35/74; A61K 36/82; A61K 36/00

(52) U.S. Cl. 424/93.4; 424/729; 424/725

(57) ABSTRACT

The present invention comprises an oral composition. More particularly to a method of treating and preventing a respiratory tract infection and providing energy, relieving stress, and mood enhancing benefits to a human comprising: the steps of administering to a human a composition comprising from about 450 IU to about 500,000 IU of cholecalciferol, per dose of the composition.

### RELATED APPLICATIONS

This application claims the benefit of U.S. Provisional Application Ser. No. 60/967,412, filed on Sep. 4, 2007.

### FIELD OF THE INVENTION

The present invention relates to an oral composition. More particularly to a method of treating and preventing a respiratory tract infection and providing energy, relief from stress, and mood enhancing benefits to a human comprising: the steps of administering to a human a composition comprising from about 450 IU to about 500,000 IU of cholecalciferol, per dose of composition.

### BACKGROUND OF THE INVENTION

Many different viruses and viral strains result in symptoms associated with respiratory illness. For example, the common cold is a complex syndrome that may be caused by any of over 200 antigenically different viruses found in several virus families. These families include rhinovirus, myxovirus, paramyxovirus, respiratory syncytial virus, adenovirus and coronavirus. Among the most important, with respect to the common cold, is rhinovirus. Although distinct from other cold viruses, the influenza virus can produce many of the same symptoms. Pinpointing the specific cause of the illness is difficult and not practical since there are also a number of predisposing factors whose contribution to the manifestation of symptoms is not fully understood.

Regardless of the virus and associated factors leading to the onset of symptoms associated with respiratory illness, a number of remedies to alleviate these symptoms have been suggested. For example, the many cough/common cold products that are currently marketed typically contain one or more of the following actives: nasal decongestants such as pseudoephedrine or oxymetazoline, antihistamines such as doxylamine; antitussives such as dextromethorphan; expectorants such as guaifenesin; and anti-pyretics such as acetaminophen. In an attempt to improve existing common cold remedies, experts in the field have suggested several alternative pharmacotherapies and have conducted common cold trials to test their efficacy.

Despite the abundance of treatments known in the art, there remains a need to provide a consistent and effective method for prevention or treatment of respiratory illness. There also remains a need to provide compositions, particularly oral compositions, and methods that are highly effective in the prevention, resistance or treatment of respiratory illness and provide less of a run-down feeling.

### SUMMARY OF THE INVENTION

The present invention comprises a method of treating and preventing a respiratory tract infection comprising: the steps of administering to a human a composition comprising from about 450 IU to about 500,000 IU of cholecalciferol, per dose of composition.

The present invention further relates to a method and compositions for providing energy, relieving stress, and mood enhancing benefits to a human comprising: the steps of administering to a human a composition comprising; (a) cholecalciferol; and (b) a tea extract.

The present invention further relates to a method of treating and preventing a respiratory tract infection comprising: the steps of administering to a human a composition comprising, (a) cholecalciferol; and (b) a probiotic.

The present invention further relates to a method of treating and preventing a respiratory tract infection comprising: the steps of administering to a human a composition comprising, (a) cholecalciferol; and (b) at least one additional component selected from the group consisting of a tea extract, Vitamin A, probiotics, Vitamin C, andrographis extract, 1-tryptophan, Allium sativum, and combinations thereof.

The present invention further relates to an oral composition for treating a respiratory tract infection in a human comprising: from about 450 IU to about 500,000 IU of cholecalciferol per dose of composition.

### DETAILED DESCRIPTION OF THE INVENTION

The present invention comprises a method for providing energy, relieving stress, and mood enhancing benefits to a human comprising: the steps of administering to a human a composition comprising; cholecalciferol; and a tea extract.

As used herein, “antimicrobial” includes antiviral, antibacterial, antifungal, antiyeast, and anti mold activities, both immediate and residual.

The term “oral compositions” as used herein refers to compositions in a form that is deliverable to the respiratory tract of the human in need. Nonlimiting examples include liquid compositions, nasal compositions, beverage, supplemental water, pills, tablets, capsules, gel compositions, sachets and combinations thereof. Nasal compositions, liquid compositions, gel compositions can be in a form that is directly deliverable to the airway passages from the nose and mouth. These compositions can be delivered via droppers, pump sprayers, irrigation devices, pressurized sprayers, atomizers, air inhalation devices and other packaging and equipment.

The oral compositions are administered to prevent and treat “cold and influenza-like symptoms”. As used herein “cold and influenza-like symptoms” refer to symptoms typically associated with respiratory tract infections. These symptoms include, but are not limited to, nasal congestion, chest congestion, sneezing, rhinorrhea, fatigue or malaise, coughing, fever, chills, body ache, sore throat, headache, and other known cold and influenza-like symptoms.

As used herein, the term “less than daily dose” and/or “less than daily schedule” includes administration of a dose of the composition every other day, once weekly, twice weekly, once every two weeks, once monthly, once every other month, and combinations thereof.

The term “respiratory viruses” as used herein refers to those viruses that are causal agents of cold and influenza-like symptoms. These viruses include Rhinovirus, Myxovirus (Influenza virus), Paramyxovirus (Parainfluenza virus), Respiratory Syncytial virus, Adenovirus and Coronavirus.

The term “safe and effective amount” of a component, composition, or like material as used herein is an amount that is effective for the treatment, prevention or resistance of respiratory illness in a mammal (preferably a human), without undue adverse side effects (such as toxicity, irritation, or allergic response), commensurate with a reasonable benefit/risk ratio when used in the manner of this invention. The specific “safe and effective amount” will, obviously, vary with such factors as the particular condition being treated, the physical condition of the treated mammal, the size and weight of the treated mammal, the duration of treatment, the nature of concurrent therapy (if any), the specific dosage form to be used, other components present in a given dosed composition, and the dosage regimen desired for the component or composition.

All weights, measurements and concentrations herein are measured at 25° C. on the composition in its entirety, unless otherwise specified.

These and other limitations of the compositions and methods of the present invention, as well as many of the optional ingredients suitable for use herein, are described in detail hereinafter.

All percentages, parts and ratios as used herein are by weight of the total composition, unless otherwise specified. All such weights as they pertain to listed ingredients are based on the active level and, therefore do not include solvents or by-products that may be included in commercially available materials, unless otherwise specified.

The composition and methods of the present invention can comprise, consist of, or consist essentially of, the essential elements and limitations of the invention described herein, as well as any additional or optional ingredients, components, or limitations described herein or otherwise useful in compositions intended for use by a mammal, preferably human use.

Composition

The present invention is a composition and methods of using the compositions by humans preferably for treating and preventing a respiratory tract infection and providing energy, relieving stress, and mood enhancing benefits. The compositions can provide replenishing energy when the human is at risk or under attack from a cold. Thereby increasing resistance to colds and flu virus and less of a run-down feeling normally associated with symptoms of a respiratory infection.

Administration of a dose of the composition may be on an as-needed or as-desired basis, or on a less than daily dosing, or daily dosing, including multiple times daily, for example, at least once daily, from one to about six times daily, from about two to about four times daily, or about three times daily.

The amount of composition administered may be dependent on a variety of factors, including the general quality of health of the mammal, age, gender, weight, or severity of symptoms.

The preferred pH range of the composition is from about 1 to about 7, from about 2 to about 6.5, from about 2 to about 5 and from about 2.6 to about 4.5.

The composition of the present invention is preferably an oral composition and can be liquid or semi-liquid, or gel or nasal compositions, beverage, supplemental water, pills, tablets, sachets or capsules.

Vitamin D

The composition of the present invention comprises Vitamin D. Nonlimiting examples of Vitamin D suitable for use in the present invention includes Vitamin D3 (cholecalciferol), Vitamin D2 (ergocalciferol) and combinations thereof. Additional, nonlimiting examples also include metabolites of Vitamin D, including calcidiol, calcitriol, and combinations thereof. The Vitamin D, including cholecalciferol, ergocalciferol, calcidiol and calcitriol, may be derived from synthetic or natural sources. Vitamin D, including cholecalciferol and calcitriol, may be sourced from an extract of solanum glaucophyllum (malacoxylon), trisetum flavescens (goldhafer) or cestrum diurnum. Both the pure, Vitamin D and/or glycosides of the Vitamin D, may be used.

Vitamin D is a unique nutrient in that its principal source is not the diet, but via synthesis in the skin upon exposure to UV light, typically sunlight in the summer months. In the skin, 7-dehydrocholesterol, derived from cholesterol, is converted by the action of UV light into Previtamin D₃ (precalciferol), which then undergoes a thermal conversion to Vitamin D₃ (cholecalciferol). Whether the cholecalciferol is synthesized in the skin or absorbed through the gut, it is transported to the liver where it is converted to 25-OH cholecalciferol (calcidiol) (25-hydroxycholecalciferol). This is the form that is ordinarily assayed in the blood. Calcidiol is eventually transported to the kidneys, where it is converted to 1,25-(OH)₂ cholecalciferol (calcitriol) the active form.

In order to provide meaningful symptom relief during the course of a respiratory tract infection and/or provide energy, relieve stress, and provide mood enhancing benefits to a human, specific blood levels of 25-hydroxycholecalciferol need to be achieved. The compositions of the present invention comprise from about 450 IU to about 500,000 IU per dose of composition, alternatively from about 500 IU to about 500,000 IU, alternatively from about 1,000 IU to about 500,000 IU of cholecalciferol, per dose of composition, alternatively from about 2,000 IU to about 100,000 IU, alternatively from about 10,000 IU to about 50,000 IU, and alternatively from about 20,000 IU to about 40,000 IU, per dose of composition.

The compositions of the present invention comprise, from about 0.45 ppm to about 125,000 ppm by weight of the composition, alternatively from about 0.5 ppm to about 125,000 ppm, alternatively from about 1 ppm to about 125,000 ppm, alternatively from about 2 ppm to about 25,000 ppm, alternatively from about 10 ppm to about 12,500 ppm, and alternatively from about 20 ppm to about 10,000 ppm by weight of composition.

With these levels of cholecalciferol administered to the human, the specific increase in blood levels of the 25-hydroxycholecalciferol in the human user can be from about 1 ng/ml to about 40 ng/ml, alternatively from about 2 ng/ml to about 30 ng/ml, alternatively from about 4 ng/ml to about 20 ng/ml, as determined by the methodology calcidiol 25-Hydroxyvitamin D ¹²⁵I RIA Kit radioimmunoassay (RIA) Catalog No./REF./KAT.-NR.:68100E manufactured, distributed and available from DiaSorin Inc., Stillwater, Minn. USA 55082.

To treat the symptoms of a respiratory tract viral infection that is already underway, the human can be administered the composition of the present invention in a daily single dose form comprising from about 450 IU to about 500,000 IU of cholecalciferol, alternatively from about 500 IU to about 500,000 IU of cholecalciferol, alternatively from about 1000 IU to about 500,000 IU of cholecalciferol, alternatively from about 5,000 IU to about 500,000 IU of cholecalciferol, alternatively from about 10,000 IU to about 100,000 IU of cholecalciferol, alternatively from about 20,000 to about 50,000 IU of cholecalciferol per dose of composition.

A daily single dose of the composition may comprise from about 0.45 ppm to about 125,000 ppm of cholecalciferol, alternatively from about 0.5 ppm to about 125,000 IU of cholecalciferol, alternatively from about 1 ppm to about 125,000 ppm of cholecalciferol, alternatively from about 5 ppm to about 125,000 ppm of cholecalciferol, alternatively from about 10 ppm to about 25,000 ppm of cholecalciferol, alternatively from about 20 ppm to about 12.500 ppm of cholecalciferol by weight of composition.

To treat or prevent the symptoms of a respiratory tract viral infection and/or provide energy, relieve stress, and provide mood enhancing benefits to a human, the human can be administered the composition of the present invention in a single dose form comprising from about 450 IU to about 10,000 IU of cholecalciferol, alternatively from about 500 IU to about 10,000 IU of cholecalciferol, alternatively from about 1,000 IU to about 5,000 IU of cholecalciferol, alternatively from about 2,000 IU to about 5,000 IU of cholecalciferol, and alternatively from about 2,000 IU to about 4,000 IU of cholecalciferol per dose of composition. The composition of the present invention in a single dose form may comprise from about 0.45 ppm to about 2500 ppm of cholecalciferol, alternatively from about 0.5 ppm to about 2500 ppm of cholecalciferol, alternatively from about 1 ppm to about 1250 ppm of cholecalciferol, alternatively from about 2 ppm to about 1250 ppm of cholecalciferol, and alternatively from about 2 ppm to about 1000 ppm of cholecalciferol by weight of composition.

In addition, meaningful symptom relief can be obtained not just from prophylactic use of cholecalciferol, but even with a treatment regimen that begins after the human is infected.

Additionally, the composition may be directly applied to the site of infection by administering a nasal composition containing calcitriol. When calcitriol is present in the nasal composition, the nasal composition comprises from about 0.000001% to about 0.0004% of calcitriol, alternatively from about 0.000002% to about 0.0003%, alternatively from about 0.000004% to about 0.0002% of calcitriol, by weight of the composition. The tissue level in the nasal passages of calcitriol when the composition is delivered directly is from about 1 pg/ml to about 40 pg/ml, alternatively from about 2 pg/ml to about 30 pg/ml, and alternatively from about 4 pg/ml to about 20 pg/ml.

The composition may also comprise Vitamin D₂ (ergocalciferol). When present the composition comprises from about 450 IU to about 500,000 IU of Vitamin D₂, per dose of composition, alternatively from about 500 IU to about 500,000 IU of Vitamin D₂, alternatively from about 1000 IU to about 500,000 IU of Vitamin D₂, alternatively from about 5,000 IU to about 500,000 IU of Vitamin D₂, per dose of composition. The composition may comprise from about 0.45 ppm to about 125,000 ppm of Vitamin D₂, per dose of composition, alternatively from about 0.500 ppm to about 125,000 ppm of Vitamin D₂, alternatively from about 1 ppm to about 125,000 ppm of Vitamin D₂, alternatively from about 5 ppm to about 125,000 ppm of Vitamin D₂, by weight of composition. With these levels of Vitamin D₂ administered to the human, the specific increase in blood levels of the 25-hydroxycholecalciferol in the human user can be from about 1 ng/ml to about 40 ng/ml, alternatively from about 2 ng/ml to about 30 ng/ml, alternatively from about 4 ng/ml to about 20 ng/ml, as determined by the methodology calcidiol 25-Hydroxyvitamin D ¹²⁵I RIA Kit radioimmunoassay (RIA) Catalog No./REF./KAT.-NR.:68100E manufactured, distributed and available from DiaSorin Inc., Stillwater, Minnesota USA 55082.

Additional Component

The composition can comprise at least one additional component selected from the group consisting of a tea extract, Vitamin A, probiotics, Vitamin C, andrographis extract, 1-tryptophan, Allium sativum, Eleutherococcus senticosus, Rhodiola rosea, Coenzyme Q10, superfruits and combinations thereof.

Tea Extract

The composition can comprise tea extract. Tea extract is a polyphenol. Nonlimiting examples of extracts includes Camellia sinensis. The tea extract has antioxidant activity so as to quench reactive oxygen species such as singlet oxygen, superoxide and hydroxyl radicals. This quenching prevents the oxidative cross-linking of test proteins and inhibits single strand breakage of DNA in whole cells; enhance antioxidant defense system by preserving antioxidant enzyme activity. The tea extract works in combination with the cholecalciferol to provide replenishing energy when the human is at risk or under attack from a cold.

Nonlimiting sources of tea extract for use in the present invention are black tea, white tea, oolong tea, and/or green tea.

When tea extract is present, the composition comprises on a dry matter basis from about 0.01% of said tea extract to about 90% of said tea extract, by weight of the composition. The composition comprising on a dry matter basis from about 0.1% of said tea extract to about 35% of said tea extract, alternatively from about 1% of said tea extract to about 15% of said tea extract, alternatively from about 1% of said tea extract to about 10% of said tea extract, alternatively from about 3% of said tea extract to about 10% of said tea extract, by weight of the composition.

When tea extract is green tea, the composition comprises on a dry matter basis from about 0.01% of said green tea extract to about 90% of said green tea extract, by weight of the composition. The composition comprising on a dry matter basis from about 0.1% of said green tea extract to about 35% of said green tea extract, alternatively from about 1% of said green tea extract to about 15% of said green tea extract, alternatively from about 1% of said green tea extract to about 10% of said green tea extract, alternatively from about 3% of said green tea extract to about 10% of said green tea extract, by weight of the composition.

Vitamin A

The composition of the present invention may comprise Vitamin A. Vitamin A and carotene can be obtained from either animal or vegetable sources. The vitamin A can be in the form of vitamin A, retinol, retinyl palmitate, retinyl acetate, retinyl propriante, beta-carotene, alpha carotene, beta-cryptoxanthin, and mixtures thereof. When Vitamin A or one of the forms of vitamin A is present, the composition comprises from about 500 IU to about 10,000 IU, alternatively from about 2,500 IU to about 10,000 IU of Vitamin A alternatively from about 3,000 IU to about 8,000 IU of Vitamin, alternatively from about 3,500 IU to about 5,000 IU of Vitamin A, per dose of composition. The composition may comprise from about 0.5 ppm to about 2500 ppm, alternatively from about 2.5 ppm to about 2500 ppm of Vitamin A, alternatively from about 3 ppm to about 2000 ppm of Vitamin A, and alternatively from about 3.500 ppm to about 1250 ppm of Vitamin A per weight of composition. The amount of Vitamin A species may be expressed as IU or as RAE (Retinol Activity Equivalent), which is equal to an equivalent amount of retinol in micrograms. For example, 10,000 IU Vitamin A is equivalent to 3000 RAE or 3000 μg retinol. Hence when Vitamin A (retinol) is present, the composition comprises from about 150 μg to about 3000 μg of retinol, alternatively from about 750 μg to about 3000 μg of retinol per dose of composition, alternatively from about 900 μg to about 2400 μg of retinol per dose of composition, alternatively from about 1050 μg to about 1500 μg of Vitamin A per dose of composition.

It is believed that over 10% of subjects with colds have suboptimal levels of Vitamin A. Vitamin A may also separately lead to the induction of Retinoic Acid Inducible Gene (RIG-I), which is a helicase sensor for double-stranded RNA that promotes an innate immune response. This response would be beneficial and complementary to the response due to the composition, in combating the symptoms of respiratory tract infections.

Probiotics

The composition of the present invention may further comprise a probiotic. When present, the composition comprises from about 10⁶ to 10¹² cfu of a probiotic, and alternatively from about 10⁶ to 10¹⁰ cfu of a probiotic. The probiotic component can be a lactic acid bacteria. Preferably the probiotic is selected from the group consisting of bacteria of the genera Bacillus, Bacteroides, Bifidobacterium, Enterococcus (e.g., Enterococcus faecium), Lactobacillus, and Leuconostoc, and combinations thereof. In another embodiment of the invention, the probiotic is selected from bacteria of the genera Bifidobacterium, Lactobacillus, and combinations thereof.

Non-limiting examples of lactic acid bacteria suitable for use herein include strains of Streptococcus lactis, Streptococcus cremoris, Streptococcus diacetylactis, Streptococcus thermophilus, Lactobacillus bulgaricus, Lactobacillus acidophilus (e.g. Lactobacillus acidophilus strain), Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus reuteri, Bifidobacterium longum, Bifidobacterium infantis, Bifidobacterium bifidum, Bifidobacterium animalis, Bifidobacterium pseudolongum, and Pediococcus cerevisiae, or mixtures thereof, preferably Lactobacillus salivarius, Bifidobacterium infantis, or mixtures thereof.

Vitamin C

The composition of the present invention may comprise Vitamin C. When Vitamin C is present, the composition comprises from about 60 mg to about 2000 mg of Vitamin C, per dose of composition, alternatively from about 80 mg to about 1500 mg of Vitamin C, per dose of composition, alternatively from about 100 mg to about 1000 mg of Vitamin C, per dose of composition. The composition may comprise from about 0.024% to about 99% of Vitamin C, alternatively from about 0.032% to about 99% of Vitamin C, alternatively from about 0.040% to about 99% of Vitamin C, per weight of composition. It is believed that Vitamin C may be of benefit in those who are under stress. Vitamin C and Vitamin D work by different and complementary mechanisms of action, the combination can provide an improved benefit in reducing the symptoms and duration of upper respiratory tract infections. It is believed that over 20% of subjects with colds have suboptimal levels of Vitamin C. The preferred form of Vitamin C for use in the composition is as ascorbic acid or the equivalent of a salt of ascorbic acid or the equivalent of a derivative of ascorbic acid. The vitamin C may either be in an immediate release form or a sustained release form.

Andrographis Paniculata

The composition may comprise an andrographis extract, an active component thereof, or mixtures thereof. As used herein, the andrographis is a plant of the genus Andrographis, having a limited number of species within this genus largely present in Asia. Only a few of the species are medicinal. In one embodiment, the plant is of the species Andrographis paniculata, which may be referenced as Kalmegh in Ayurvedic medicine.

Andrographis is typically standardized by quantifying the total amount of andrographolides, which often make up 5 to 20% of the extract.

When present the composition comprises andrographis containing from about 5 mg to about 50 mg of andrographolides, per dose of composition, alternatively from about 10 mg to about 40 mg of andrographolides, per dose of composition, alternatively from about 15 mg to about 30 mg of andrographolides per dose of composition. The composition may comprise andrographis containing from about 0.002% to about 50% of andrographolides, alternatively from about 0.004% to about 40% of andrographolides, alternatively from about 0.006% to about 30% of andrographolides per weight of composition.

When present the composition comprises Andrographis paniculata containing from about 5 mg to about 50 mg of andrographolides, per dose of composition, alternatively from about 10 mg to about 40 mg of andrographolides, per dose of composition, alternatively from about 15 mg to about 30 mg of andrographolides, per dose of composition. The composition may comprise andrographis that contains from about 0.002% to about 50% of andrographolides, alternatively from about 0.004% to about 40% of andrographolides, alternatively from about 0.006% to about 30% of andrographolides per weight of composition.

Andrographis aids in reducing to an extent the symptoms or duration of colds. It is believed that andrographis paniculata has a similar mechanism of action to Vitamin D, whereby andrographis paniculata reduces the levels of inflammatory cytokines and chemokines, such as IP-10. Andrographolide, the principal component of andrographis, is remarkably similar in chemical structure to Vitamin D. Therefore, andrographis may provide some of its benefits by acting as a ligand at the Vitamin D receptor.

By combining andrographis with the composition provide additional benefits including but not limited to improved onset of relief as andrographis is metabolized differently than Vitamin D, and improved duration of relief as Vitamin D has a much longer half-life in the blood than does andrographis.

l-Tryptophan

The composition of the present invention may comprise l-tryptophan. When 1-tryptophan is present, the composition comprises from about 250 mg to about 2500 mg of l-tryptophan, per dose of composition, alternatively from about 300 mg to about 2000 mg of l-tryptophan, per dose of composition, alternatively from about 400 mg to about 1000 mg of l-tryptophan, per dose of composition. The composition may comprise from about 0.1% to about 99% of l-tryptophan, alternatively from about 0.12% to about 99% of l-tryptophan, alternatively from about 0.16% to about 99% of l-tryptophan, by weight of composition. Not to be limited by theory, the inflammatory cytokines and chemokines produced during a cold may be responsible for the malaise and fatigue associated with colds through their interaction with the olfactory bulb of the nasal passages, and ultimately with the Hypothalamus-Pituitary-Adrenal (HPA) axis. The HPA axis is the seat of many physiological functions, playing a major role in controlling mood. Serotonin is a mediator that acts on the HPA axis and is correlated with mood. Tryptophan, the metabolic precursor to serotonin, is degraded by cytokines that are unleashed during an upper respiratory tract viral infection. Therefore tryptophan levels may be lowered during a cold, leading to worsened mood, malaise and a feeling of fatigue.

A composition comprising l-tryptophan can provide a mechanism by which the HPA axis could improve the malaise and fatigue associated with cold, and hence provide a meaningful benefit in the relief of colds symptoms.

Allium Sativum

The composition may comprise Allium Sativum (garlic). Allium Sativum has been shown to be effective at reducing many of the cytokines and chemokines involved in the immune response to viral infections. Therefore, Allium Sativum can improve the symptoms of colds and flu by reducing the inflammatory cytokines and chemokines that have been shown to play a major role in producing symptoms. A combination of Allium Sativum, and/or Allicin, a component of Allium Sativum, with the composition should provide extensive relief of colds and flu symptoms. When present the composition comprises on a dry matter basis from about 0.01% of said Allium Sativum to about 90% of said Allium Sativum, by weight of the composition. The composition comprising on a dry matter basis from about 0.1% of said Allium Sativum to about 35% of said Allium Sativum, alternatively from about 1% of said Allium Sativum to about 15% of said Allium Sativum, alternatively from about 1% of said Allium Sativum to about 10% of said Allium Sativum, alternatively from about 3% of said Allium Sativum to about 10% of said Allium Sativum, by weight of the composition.

The composition may comprise from about 100 mg to about 10,000 mg of Allium sativum, per dose of composition, alternatively from about 200 mg to about 5000 mg, alternatively from about 500 mg to about 2000 mg of Allium sativum, per dose of composition.

The composition may comprise from about 1000 μg to about 100,000 μg of Allicin, per dose of composition, alternatively from about 2000 μg to about 50,000 μg, alternatively from about 5000 μg to about 20,000 μg of Allicin, per dose of composition.

The composition may comprise from about 4 ppm to about 99% of Allicin, per dose of composition, alternatively from about 8 ppm to about 50%, alternatively from about 20 ppm to about 20% of Allicin, by weight of composition.

Eleutherococcus Senticosus

The composition may comprise Eleutherococcus senticosus. Eleutherococcus senticosus, also known as Acanthopanax senticosus, and commonly called Siberian ginseng, is an herb traditionally used for symptoms of asthenia such as fatigue and weakness. Eleutherococcus is thought to have adaptogenic properties that will provide complementary relief to that derived from Vitamin D, especially with regards to providing an energy benefit. The active ingredients of this plant are typically concentrated in the root and mainly consist of chemically distinct glycosides called eleutherosides.

When present the composition may comprise from about 100 mg to about 10,000 mg of Eleutherococcus root, per dose of composition, alternatively from about 200 mg to about 5000 mg, alternatively from about 2000 mg to about 3000 mg of Eleutherococcus root, per dose of composition. The composition may comprise from about 0.04% to about 99% mg of Eleutherococcus root, per dose of composition, alternatively from about 0.08% to about 99%, alternatively from about 0.80% to about 99% of Eleutherococcus root, by weight of composition.

In an alternative embodiment, a dried hydroalcoholic extract of eleutherococcus root may be used, in which about 65 mg to about 195 mg of extract corresponds to about 2 g to about 3 g of the whole root. The composition may comprise from about 3 mg to about 650 mg of eleutherococcus extract, per dose of composition, alternatively from about 6 mg to about 325 mg, alternatively from about 65 mg to about 195 mg of eleutherococcus extract, per dose of composition. The composition may comprise from about 0.001% to about 99% of eleutherococcus extract, alternatively from about 0.002% to about 99%, alternatively from about 0.026% mg to about 99% of eleutherococcus extract, by weight of composition.

Rhodiola Rosea

The composition may further comprise Rhodiola rosea, also known as golden root. Rhodiola rosea is an herb that is traditionally used for improving mood and alleviating depression. Rhodiola is thought to have adaptogenic properties that will provide complementary relief to that derived from Vitamin D, especially with regards to providing an energy benefit. Extracts of rhodiola root commonly contain from about 2% to about 3% of rosavins and salidroside.

When present the composition may comprise from about 10 mg to about 1000 mg of rhodiola extract, per dose of composition, alternatively from about 100 mg to about 500 mg, alternatively from about 250 mg to about 400 mg of rhodiola extract, per dose of composition. The composition may comprise from about 0.004% to about 99% of rhodiola extract, alternatively from about 0.04% to about 99%, alternatively from about 0.10% to about 99% of rhodiola extract, by weight of composition.

Coenzyme Q10

The composition may further comprise Coenzyme Q10, also known as ubiqinone. Coenzyme Q10 is a compound found naturally in mitochondria, the energy-producing center of the cell. CoQ10 is required for energy production and is known to decline with age. CoQ10 is involved in the process of manufacturing ATP, which serves as the cell's major energy source and drives a number of biological processes including muscle contraction and the production of protein. CoQ10 can also function as an antioxidant.

When present the dosage units comprise at least about 0.01%, alternatively from about 0.01% to about 10%, and alternatively from about 0.2% to about 5% Coenzyme Q10, by weight of the composition of the dosage unit.

The composition can comprise from about 1 mg to about 400 mg, alternatively from about 2 mg to about 400 mg, and alternatively from about 3 mg to about 300 mg of Coenzyme Q10, per dosage unit.

Superfruits

The composition can comprise superfruits. extracts of certain superfruits have substantial antioxidant and other health benefits. These superfruits have exceptional nutrient richness and antioxidant quality with appealing taste. Nonlimiting examples of Superfruits include Açaí, Blueberry, Cranberry, Grape, Guarana, Mangosteen, Noni, Pomegranate (Punica granatum), Seabuckthorn, Wolfberry (Goji), acerola (Barbados cherry, Malpighia emarginata, Malpighia glabra), bayberry (yumberry, Myrica rubra), bilberry (Vaccinium myrtillus), black raspberry (Rubus occidentalis), black chokeberry (“aronia”, Aronia melanocarpa), blackcurrant (Ribes nigrum), camu camu (Myrciaria dubia), sour (tart) cherry (Prunus cerasus), cupuaçu (Theobroma grandiflorum), durian (Durio kutejensis), elderberry (Sambucus canadensis, Sambucus nigra), red guava (Psidium guajava, many species), Indian gooseberry (amalaka, amla, Phyllanthus emblica), kiwifruit (Actinidia deliciosa), lingonberry (Vaccinium vitis-idaea), lychee (Litchi chinensis), muscadine grape (Vitis rotundifolia), papaya (Carica papaya), pomelo (Citrus maxima), saskatoon berry (Amelanchier alnifolia, Nutt), tamarind (Tamarindus indica), wild cherry (Prunus avium) andyuzu (Citrus ichangensis, C. reticulata) and combinations thereof.

Punica granatum contains a class of polyphenolic tannins called punicalagins, which are made of smaller acids, such as ellagic acid and gallic acid. These polyphenols can act in a complementary fashion to Vitamin D to provide relief from symptoms of respiratory tract infections.

The composition can comprise at least about 0.01%, alternatively from about 0.01% to about 10%, and alternatively from about 0.2% to about 5% of a superfruit extract, by weight of the composition of the dosage unit.

The composition can comprise from about 1 g to about 3000 mg, alternatively from about 10 mg to about 2000 mg, and alternatively from about 50 mg to about 1000 mg of a superfruit extract, per dosage unit.

The composition can comprise at least about 0.01%, alternatively from about 0.01% to about 10%, and alternatively from about 0.2% to about 5% Punica granatum extract, by weight of the composition of the dosage unit.

The composition can comprise from about 1 mg to about 3000 mg, alternatively from about 10 mg to about 2000 mg, and alternatively from about 50 mg to about 1000 mg of Punica granatum extract, per dosage unit.

Respiratory Ingredients

The compositions of the present invention can comprise a wide range of respiratory ingredients. Nonlimiting examples include analgesics, anticholinergics, antihistamines, anti-inflammatories, antipyretics, antitussives, antivirals, decongestants, expectorants, mucolytics, and combinations thereof.

Example decongestants include: oxymetazoline, phenylephrine, xylometazoline, naphazoline, l-desoxyephedrine, ephedrine, propylhexedrine, pseudoephedrine, and phenylpropanolamine. Example anticholinergics include: ipratropium, chlorpheniramine, brompheniramine, diphenhydramine, doxylamine, clemastine, and triprolidine. Common analgesics, anti-inflammatories and antipyretics include: ibuprofen, ketoprofen, diclofenac, naproxen, acetaminophen, and aspirin. Example antivirals include: amantidine, rimantidine, pleconaril, zanamivir, and oseltamivir. Examples of antitussives include codeine, dextromethorphan, chlophedianol and levodropropizine. Examples of expectorants include guaifenesin. Examples of mucolytics include ambroxol and N-acetylcysteine. Examples of antihistamines include diphenhydramine, doxylamine, triprolidine, clemastine, pheniramine, chlorpheniramine, brompheniramine, loratadine, cetirizine and fexofenadine.

Wherein a respiratory ingredient is utilized herein, the compositions may optionally comprise from about 0.001% to about 10% of the additional ingredient, by weight of the composition.

Carrier

The compositions of the present invention may be administered orally as compositions comprising a pharmaceutically acceptable carrier system. Any pharmaceutically acceptable carrier in the form of a liquid, solid, or gas is suitable for the delivery of the compositions to prevent and treat common cold, influenza-like, and allergy symptoms. Depending on the desired dose form of the composition and, where applicable, the delivery device to be used, the compositions of the present invention may optionally include a pharmaceutically acceptable carrier such as water, water-miscible solvents including ethanol, propylene glycol, polyethylene glycol, transcutol, glycerol, and other known or otherwise effective water-miscible solvents; liquid aerosol propellants; and mixtures thereof. In one embodiment, these carriers are isotonic with human plasma. When the compositions of the present invention are administered using water as a pharmaceutically acceptable carrier, the water may optionally be purified or de-ionized water, and may be substantially free of organic impurities and/or meets the USP guidelines for purified water. The concentration of water utilized to formulate the compositions as a final product form for delivery to respiratory tract areas may range from about 0% to about 99.85%, from about 10% to about 90%, alternatively from about 20% to about 80%, alternatively from about 25% to about 75%, by weight of the composition. When the compositions of the present invention are administered using a solid pharmaceutically acceptable carrier, the carrier may be dosed as a powder, a capsule or tablet form. Pharmaceutically acceptable solid carriers can be added to provide aid in processing of the compositions, to aid in the consistency of the compositions, to provide for improved stability, to facilitate handling, for hygroscopicity benefits, and so forth. Pharmaceutically acceptable solid carrier materials include ingredients such as particulate and powder fillers, for example, a lactose powder, a sucrose powder and/or mixtures thereof.

Sweetener

As another non-limiting example, the composition may comprise a sweetener. Some natural and artificial sweeteners usable with the present invention include but are not limited to glucose, fructose, saccharine and its salts, sucrose, cyclamates, xylatols, acesulfame K, sucralose, and aspartame.

Optional Ingredients

The compositions of the present invention can comprise a wide range of optional ingredients. Nonlimiting examples of optional ingredients include antimicrobial metal salts, optional mildness enhancers, optional stabilizers, abrasives, antioxidants, biological additives, chemical additives, colorants, coolants, chelants, denaturants, drug astringents, emulsifiers, external analgesics, film formers, fragrance compounds, humectants, opacifying agents, plasticizers, preservatives, propellants, reducing agents, solvents, foam boosters, hydrotropes, solublizing agents, suspending agents (non-surfactant), a solvent, viscosity increasing agents (aqueous and non-aqueous), sequestrants, vitamins, antioxidants, buffers, keratolytics, and the like, and combinations thereof. Preferably the optional ingredient is selected from the group consisting of solvents, a chelant, a preservative, a fragrance, buffer, antimicrobial metal salts and combinations thereof.

Nonlimiting examples of antimicrobial metal salts include zinc, iron, copper, silver, tin, bismuth, and combinations thereof.

Nonlimiting examples of preservative include but are not limited to benzoalkonium chloride, EDTA, benzyl alcohol, potassium sorbate, parabens, and mixtures thereof.

Unless otherwise specified, the compositions may optionally comprise one or more given optional ingredients at concentrations ranging from about 0.001% to about 99%, alternatively from about 0.01% to about 80%, alternatively from about 0.01% to about 50%, alternatively from about 0.01% to about 10%, all by weight of the composition.

Method of Making

The compositions of the present invention may be prepared by any known or otherwise effective techniques suitable for providing a composition that provides a therapeutic benefit in the prevention and treatment of common cold, influenza-like, and allergy symptoms. The compositions are preferably formulated to comprise a cholecalciferol and/or calcitroil and/or a green tea extract, wherein these compositions may then be manufactured into final product forms of liquids, sprays, powders, capsules, semi-liquid, gel, nasal compositions, beverage, supplemental water, pills, tablets or the like for oral ingestion and absorption to prevent or treat symptoms associated with respiratory illness.

Methods of Use

The present invention is directed to methods of treating, preventing a respiratory illness and/or providing energy, relieving stress, and mood enhancing benefits comprising orally administering a composition as described herein to a mammal in need of such treatment. As used herein, the term “treating”, with reference to the respiratory illness refers to preventing, alleviating a symptom, or curing. As used herein, the term “respiratory illness” encompasses a broad range of respiratory ailments, including viral infections such as influenza and common cold, as well as allergy. Respiratory illness may present as any of a variety of symptoms, such as runny nose, sneezing, rhinorrhea, nasal congestion, chest congestion, cough, pressure, headache, body ache, fever, sore throat, fatigue, chills, and the like. The mammal treated may be a human, or a companion animal such as a dog, cat or horse.

As used herein the term “orally administering” with respect to the mammal means that the mammal ingests or is directed to ingest, or does ingest, or deliver, one or more of the present compositions. Wherein the human is directed to ingest or deliver the composition, such direct and or deliver may be that which instructs and/or informs the human that use of the composition may and/or will provide relief from the respiratory illness (e.g., symptomatic relief, whether temporary or permanent) for example, relief from congestion. For example, such direction may be oral direction (e.g., through oral instruction from, for example, a physician, pharmacist, or other health professional), radio or television media (e.g., advertisement), or written direction (e.g., through written direction from, for example, a physician, pharmacist, or other health professional (e.g., scripts), sales professional organization (e.g., through, for example, marketing brochures, pamphlets, or other instructive paraphernalia), written media (e.g., internet, electronic mail, or other computer-related media)), and/or packaging associated with the composition (e.g., a label present on a container holding the composition). As used herein “written” means through words, pictures, symbols, and/or other visible or tactile descriptors. Such information need not utilize the actual words used herein, for example, “respiratory”, “illness”, or “mammal”, but rather use of words, pictures, symbols, tactile means, and the like conveying the same or similar meaning are contemplated within the scope of this invention.

In one embodiment herein, an oral composition is administered to the mammal, that provides in total dosage amounts, per dose, of from about 0.15 to about 180 μg/kg body weight of the mammal, alternatively from about 0.7 to about 40 μg/kg body weight of the mammal, alternatively from about 3 to about 20 μg/kg body weight of the mammal, of cholecalciferol.

In one embodiment herein, an oral composition is administered to the mammal, that provides in total dosage amounts, per dose, of from about 10 to about 1500 μg/kg body weight of the mammal, alternatively from about 50 to about 750 μg/kg body weight of the mammal, alternatively from about 100 to about 300 μg/kg body weight of the mammal, of cholecalciferol and green tea extract.

In one embodiment herein, a nasal composition is administered to the mammal, that provides in total dosage amounts, per dose, of from about 50 to about 2000 μg/pg body weight of the mammal, alternatively from about 100 to about 1500 μg/pg body weight of the mammal, alternatively from about 200 to about 1000 μg/pg body weight of the mammal, of calcitriol. The local tissue level of 25-hydroxycholecalciferol in the nasal passages once calcitriol has been delivered directly is from about 1 pg/ml to about 40 pg/ml, alternatively from about 2 pg/ml to about 30 pg/ml, and alternatively from about 4 pg/ml to about 20 pg/ml of 25-hydroxycholecalciferol as determined by the methodology calcidiol 25-Hydroxyvitamin D ¹²⁵I RIA Kit radioimmunoassay (RIA) Catalog No./REF./KAT.-NR.:68100E manufactured, distributed and available from DiaSorin Inc., Stillwater, Minnesota USA 55082.

### EXAMPLES

The following examples further describe and demonstrate embodiments within the scope of the invention. The examples are given solely for the purpose of illustration and are not to be construed as limitations of the present invention, as many variations thereof are possible without departing from the spirit and scope of the invention.

### Example No. 1

### A Capsule Containing 2000 IU Cholecalciferol

| Ingredient | Amount per Capsule |
|---|---|
| Vitamin D3 | 50 μg |
| (cholecalciferol) | |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the other excipients. The ingredients are accurately dosed into a hard gelatin capsule. One or two capsules may be taken daily during the colds season.

### Example No. 2

### A Capsule Containing 5000 IU Cholecalciferol

| Ingredient | Amount per Capsule |
|---|---|
| Vitamin D3 | 125 μg |
| (cholecalciferol) | |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the other excipients. The ingredients are accurately dosed into a hard gelatin capsule. One or two capsules may be taken daily while the patient has a cold or influenza.

### Example No. 3

### A Tablet Containing 2000 IU Cholecalciferol

| Ingredient | Amount per Tablet | |
|---|---|---|
| Vitamin D3 | 50 | μg |
| (cholecalciferol) | | |
| Calcium Carbonate | 450 | mg |
| Cellulose Gel | 10 | mg |
| Croscarmellose | 10 | mg |
| Sodium | | |
| Magnesium Stearate | 2 | mg |

The Vitamin D3 is granulated with the calcium carbonate and cellulose gel. After granulation, the powder is dried and milled. The dried, milled powder is mixed with the croscarmellose sodium and then with the magnesium stearate. The blended powder is the compressed on a tablet press. One or two tablets may be taken daily during the colds season.

### Example No. 4

### A Tablet Containing 5000 IU Cholecalciferol

| Ingredient | Amount per Tablet | |
|---|---|---|
| Vitamin D3 | 125 | μg |
| (cholecalciferol) | | |
| Calcium Carbonate | 450 | mg |
| Cellulose Gel | 10 | mg |
| Croscarmellose | 10 | mg |
| Sodium | | |
| Magnesium Stearate | 2 | mg |

The Vitamin D3 is granulated with the calcium carbonate and cellulose gel. After granulation, the powder is dried and milled. The dried, milled powder is mixed with the croscarmellose sodium and then with the magnesium stearate. The blended powder is the compressed on a tablet press. One or two tablets may be taken daily while the patient has a cold or influenza.

### Example No. 5

### A Nasal Spray/Composition Containing 0.0004% Calcitriol

| Ingredient | % w/v |
|---|---|
| Calcitriol | 0.0004% |
| Ethanol | 5.0% |
| Hydroxypropyl | 2.00% |
| methylcellulose | |
| Monosodium | 0.05% |
| phosphate, | |
| monohydrate | |
| Disodium phosphate, | 0.17% |
| heptahydrate | |
| Benzalkonium | 0.40% |
| chloride | |
| Disodium edentate | 0.05% |
| Fragrance | q.s. |
| Purified water | q.s to 100% |

The calcitriol may be dissolved in the ethanol and then mixed with the other ingredients. One or two sprays per nostril may be taken once or twice daily while the patient has a cold or influenza.

### Example No. 6

### A Tablet Containing 2000 IU Cholecalciferol and 2500 IU Retinol

| Ingredient | Amount per Tablet | |
|---|---|---|
| Vitamin D3 | 50 | μg |
| (cholecalciferol) | | |
| Vitamin A | 750 | μg |
| (retinol) | | |
| Calcium Carbonate | 450 | mg |
| Cellulose Gel | 10 | mg |
| Croscarmellose | 10 | mg |
| Sodium | | |
| Magnesium Stearate | 2 | mg |

The Vitamin D3 and Vitamin A is granulated with the calcium carbonate and cellulose gel. After granulation, the powder is dried and milled. The dried, milled powder is mixed with the croscarmellose sodium and then with the magnesium stearate. The blended powder is the compressed on a tablet press. One or two tablets may be taken daily during the colds season.

### Example No. 7

### A Tablet Containing 2000 IU Cholecalciferol and 60 mg Ascorbic Acid

| Ingredient | Amount per Tablet | |
|---|---|---|
| Vitamin D3 | 50 | μg |
| (cholecalciferol) | | |
| Vitamin C | 60 | mg |
| (ascorbic acid) | | |
| Calcium Carbonate | 450 | mg |
| Cellulose Gel | 10 | mg |
| Croscarmellose | 10 | mg |
| Sodium | | |
| Magnesium Stearate | 2 | mg |

The Vitamin D3 and Vitamin C is granulated with the calcium carbonate and cellulose gel. After granulation, the powder is dried and milled. The dried, milled powder is mixed with the croscarmellose sodium and then with the magnesium stearate. The blended powder is the compressed on a tablet press. One or two tablets may be taken daily during the colds season.

### Example No. 8

### A Capsule Containing Vitamin D and Probiotic Bacteria Amounts:

| Ingredient | Amount per Capsule |
|---|---|
| Bifidobacterium | 1% |
| infantis | |
| Microcrystalline | 93.975% |
| cellulose | |
| Magnesium Stearate | 5% |
| Vitamin D3 | .025% |
| (cholecalciferol) | |

These components are filled in a hydroxypropyl methyl cellulose (HPMC) capsule to an approximate fill weight of 200 mg.

### Example No. 9

### A Capsule Containing 5000 IU Cholecalciferol and Andrographis Paniculata Extract

| Ingredient | Amount per Capsule |
|---|---|
| Vitamin D3 | 125 μg |
| (cholecalciferol) | |
| Andrographis | 300 mg |
| Paniculata Extract | |
| (10% | |
| andrographolides) | |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the andrographis paniculata extract and the other excipients. The ingredients are accurately dosed into a hard gelatin capsule. One or two capsules may be taken daily while the patient has a cold or influenza.

### Example No. 10

### A capsule Containing 5000 IU Cholecalciferol and Tryptophan

| Ingredient | Amount per Capsule |
|---|---|
| Vitamin D3 | 125 μg |
| (cholecalciferol) | |
| Tryptophan | 250 mg |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the tryptophan and the other excipients. The ingredients are accurately dosed into a hard gelatin capsule. One or two capsules may be taken daily while the patient has a cold or influenza.

### Example No. 11

### A Capsule Containing 5000 IU Cholecalciferol and Allium Sativum

| Ingredient | Amount per Capsule |
|---|---|
| Vitamin D3 | 125 μg |
| (cholecalciferol) | |
| Allium sativum | 180 mg |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the allium sativum and the other excipients. The ingredients are accurately dosed into a hard gelatin capsule. One or two capsules may be taken daily while the patient has a cold or influenza.

### Example No. 12

### A Capsule Containing 5000 IU Cholecalciferol, 325 mg Acetaminophen, 25 mg Dextromethorphan and 30 mg Pseudoephedrine

| Ingredient | Amount per Capsule |
|---|---|
| Vitamin D3 | 125 μg |
| (cholecalciferol) | |
| Acetaminophen | 325 mg |
| Dextromethorphan | 25 mg |
| HBr | |
| Pseudoephedrine HCl | 30 mg |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose or spray-dried onto the cellulose. The diluted Vitamin D3 is then mixed with the acetaminophen, dextromethorphan, pseudoephedrine and the other excipients. The ingredients are accurately dosed into a hard gelatin capsule. One or two capsules may be taken daily while the patient has a cold or influenza.

### Example No. 13

### A Liquid Composition Containing 2000 IU Cholecalciferol Per Dose

| Ingredient | Amount | |
|---|---|---|
| Vitamin D3 | 333 | μg |
| (cholecalciferol) | | |
| Ethanol (95%) | 33.3 | mg |
| Preservative | 0.1 | g |
| Flavor | 0.1 | g |
| Color | 0.01 | g |
| Water | QS to 100 | ml |

The Vitamin D3 may be dissolved in the ethanol. The Vitamin D3 solution is then mixed with the other excipients. A dose would be 15 ml.

### Example No. 14

### A Solid Composition Containing 1000 IU Cholecalciferol Per Dose

| Ingredient | Amount | |
|---|---|---|
| Thiamine (B1) | 2.8 | mg |
| Riboflavin (B2) | 3.2 | mg |
| Niacin (B3) | 5 | mg |
| Pantothenic Acid (B5) | 6 | mg |
| Pyridoxine (B6) | 5 | mg |
| Biotin (B7) | 160 | μg |
| Folic Acid (B9) | 400 | μg |
| Cobalamin (B12) | 3 | μg |
| Ascorbic Acid (C) | 300 | mg |
| Cholecalciferol (D3) | 12.5 | μg |
| Green Tea | 5.00% | |
| (10% caffeine) | | |
| Sucrose | 200 | mg |

| | |
|---|---|
| Flavor | QS |
| Color | QS |

The Vitamins may be premixed and granulated with the sucrose. The powder may be packaged in sachets. A dose may be two sachets.

### Example No. 15

### A Solid Composition Containing 1000 IU Cholecalciferol Per Dose

| Ingredient | Amount | |
|---|---|---|
| Thiamine (B1) | 2.8 | mg |
| Riboflavin (B2) | 3.2 | mg |
| Pyridoxine (B6) | 3.23 | mg |
| Cobalamin (B12) | 3 | μg |
| Ascorbic Acid (C) | 300 | mg |
| Cholecalciferol (D3) | 12.5 | μg |
| Siberian ginseng | 64 | mg |
| Sucrose | 500 | mg |

| | |
|---|---|
| Flavor | QS |
| Color | QS |

The Vitamins may be premixed and granulated with the sucrose. The powder may be packaged in sachets. A dose may be two sachets.

### Example No. 16

### A Capsule Containing 5000 IU Cholecalciferol and Rhodiola Rosea

| Ingredient | Amount per Capsule |
|---|---|
| Vitamin D3 | 125 μg |
| (cholecalciferol) | |
| Rhodiola rosea | 360 mg |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the rhodiola rosea and the other excipients. The ingredients are accurately dosed into a hard gelatin capsule. One or two capsules may be taken daily while the patient has a cold or influenza.

### Example No. 17

### A Capsule Containing 5000 IU Cholecalciferol and Coenzyme Q10

| Ingredient | Amount per Capsule |
|---|---|
| Vitamin D3 | 125 μg |
| (cholecalciferol) | |
| Coenzyme Q10 | 300 mg |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the Coenzyme Q10 and the other excipients. The ingredients are accurately dosed into a hard gelatin capsule. One or two capsules may be taken daily while the patient has a cold or influenza.

### Example No. 18

### A Capsule Containing 5000 IU Cholecalciferol and Punica Granatum

| Ingredient | Amount per Capsule |
|---|---|
| Vitamin D3 | 125 μg |
| (cholecalciferol) | |
| Punica granatum | 80 mg |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the Punica granatum and the other excipients. The ingredients are accurately dosed into a hard gelatin capsule. One or two capsules may be taken daily while the patient has a cold or influenza.

### Example No. 19

### A Capsule Containing 2000 IU Ergocalciferol

| Ingredient | Amount per Capsule |
|---|---|
| Vitamin D2 | 50 μg |
| (ergocalciferol) | |
| Magnesium Stearate | 5 mg |
| Silicon Dioxide | 2 mg |
| Microcrystalline | q.s. to Volume |
| Cellulose | |

The Vitamin D2 may be diluted serially in the cellulose, or granulated with the cellulose, spray-dried onto the cellulose. The diluted Vitamin D2 is then mixed with the other excipients. The ingredients are accurately dosed into a hard gelatin capsule. One or two capsules may be taken daily during the colds season.

### Example No. 20

### A Capsule Containing 2000 IU Cholecalciferol and Green Tea

| Ingredient | Amount per Capsule |
|---|---|
| Vitamin D3 | 50 μg |
| (cholecalciferol) | |
| Green Tea (10% | 50 mg |
| caffeine) | |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the Green Tea and other excipients. The ingredients are accurately dosed into a hard gelatin capsule. One or two capsules may be taken daily during the winter or other times when the subject is expected to have low Vitamin D levels to increase energy, relieve stress or enhance overall mood.

### Example No. 21

### A Capsule Containing 5000 IU Cholecalciferol and Eleutherococcus Senticosus

| Ingredient | Amount per Capsule |
|---|---|
| Vitamin D3 | 125 μg |
| (cholecalciferol) | |
| Eleutherococcus | 64 mg |
| senticosus extract | |
| Magnesium Stearate | 5 mg |
| Silicon Dioxide | 2 mg |
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the eleutherococcus senticosus extract and other excipients. The ingredients are accurately dosed into a hard gelatin capsule. One or two capsules may be taken daily while the patient has a cold or influenza.

### Example No. 22

### A Tablet Containing 2000 IU Cholecalciferol and Eleutherococcus Senticosus

| Ingredient | Amount per Tablet | |
|---|---|---|
| Vitamin D3 | 50 | μg |
| (cholecalciferol) | | |
| Eleutherococcus | 64 | mg |
| senticosus extract | | |
| Calcium Carbonate | 450 | mg |
| Cellulose Gel | 10 | mg |
| Croscarmellose | 10 | mg |
| Sodium | | |
| Magnesium Stearate | 2 | mg |

The Vitamin D3 is granulated with the calcium carbonate and cellulose gel. After granulation, the powder is dried and milled. The dried, milled powder is mixed with the Eleutherococcus senticosus extract and the croscarmellose sodium and then with the magnesium stearate. The blended powder is the compressed on a tablet press. One or two tablets may be taken daily during the winter or other times when the subject is expected to have low Vitamin D levels to increase energy, relieve stress or enhance overall mood.

### Example No. 23

### A Tablet Containing 5000 IU Cholecalciferol and Green Tea

| Ingredient | Amount per Tablet | |
|---|---|---|
| Vitamin D3 | 125 | μg |
| (cholecalciferol) | | |
| Green Tea (8% | 50 | mg |
| caffeine) | | |
| Calcium Carbonate | 450 | mg |
| Cellulose Gel | 10 | mg |
| Croscarmellose | 10 | mg |
| Sodium | | |
| Magnesium Stearate | 2 | mg |

The Vitamin D3 is granulated with the calcium carbonate and cellulose gel. After granulation, the powder is dried and milled. The dried, milled powder is mixed with the Green Tea and the croscarmellose sodium and then with the magnesium stearate. The blended powder is the compressed on a tablet press. One or two tablets may be taken daily during winter or other times when the subject is expected to have low Vitamin D levels to increase energy, relieve stress or enhance overall mood.

### Example No. 24

### A Tablet Containing 2000 IU Cholecalciferol, 2500 IU Retinol and Green Tea

| Ingredient | Amount per Tablet | |
|---|---|---|
| Vitamin D3 | 50 | μg |
| (cholecalciferol) | | |
| Vitamin A | 750 | μg |
| (retinol) | | |
| Green Tea (10% | 300 | mg |
| caffeine) | | |
| Calcium Carbonate | 450 | mg |
| Cellulose Gel | 10 | mg |
| Croscarmellose | 10 | mg |
| Sodium | | |
| Magnesium Stearate | 2 | mg |

The Vitamin D3 and Vitamin A is granulated with the calcium carbonate and cellulose gel. After granulation, the powder is dried and milled. The dried, milled powder is mixed with the green tea and the croscarmellose sodium and then with the magnesium stearate. The blended powder is the compressed on a tablet press. One or two tablets may be taken daily during winter or other times when the subject is expected to have low Vitamin D levels to increase energy, relieve stress or enhance overall mood.

### Example No. 25

### A Tablet Containing 2000 IU Cholecalciferol, 60 mg Ascorbic Acid and Eleutherococcus Senticosus

| Ingredient | Amount per Tablet | |
|---|---|---|
| Vitamin D3 | 50 | μg |
| (cholecalciferol) | | |
| Vitamin C | 60 | mg |
| (ascorbic acid) | | |
| Eleutherococcus | 64 | mg |
| Senticosus Extract | | |
| Calcium Carbonate | 450 | mg |
| Cellulose Gel | 10 | mg |
| Croscarmellose | 10 | mg |
| Sodium | | |
| Magnesium Stearate | 2 | mg |

The Vitamin D3 and Vitamin C is granulated with the calcium carbonate and cellulose gel. After granulation, the powder is dried and milled. The dried, milled powder is mixed with the Eleutherococcus senticosus extract and the croscarmellose sodium and then with the magnesium stearate. The blended powder is the compressed on a tablet press. One or two tablets may be taken daily during winter or other times when the subject is expected to have low Vitamin D levels to increase energy, relieve stress or enhance overall mood.

### Example No. 26

### A Capsule Containing Vitamin D, Probiotic Bacteria and Green Tea Amounts:

| Ingredient | Amount per Capsule |
|---|---|
| Bifidobacterium | 1% |
| infantis | |
| Microcrystalline | 83.975% |
| cellulose | |
| Green Tea (8% | 10% |
| caffeine) | |
| Magnesium Stearate | 5% |
| Vitamin D3 | .025% |
| (cholecalciferol) | |

These components are filled in a hydroxypropyl methyl cellulose (HPMC) capsule to an approximate fill weight of 200 mg. One or two capsules may be taken daily during winter or other times when the subject is expected to have low Vitamin D levels to increase energy, relieve stress or enhance overall mood.

### Example No. 27

### A Capsule Containing 5000 IU Cholecalciferol, Andrographis Paniculata Extract and Eleutherococcus Senticosus Extract

| Ingredient | Amount per Capsule | |
|---|---|---|
| Vitamin D3 | 125 | μg |
| (cholecalciferol) | | |
| Andrographis | 300 | mg |
| Paniculata Extract | | |
| (10% andrographolides) | | |
| Eleutherococcus | 64 | mg |
| Senticosus Extract | | |
| Magnesium Stearate | 5 | mg |
| Silicon Dioxide | 2 | mg |

| | |
|---|---|
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the andrographis paniculata extract, eleutherococcus senticosus extract and the other excipients. The ingredients are accurately dosed into a hard gelatin capsule.

One or two capsules may be taken daily during winter or other times when the subject is expected to have low Vitamin D levels to increase energy, relieve stress or enhance overall mood.

### Example No. 28

### A Capsule Containing 5000 IU Cholecalciferol, Tryptophan and Green Tea

| Ingredient | Amount per Capsule | |
|---|---|---|
| Vitamin D3 | 125 | μg |
| (cholecalciferol) | | |
| Tryptophan | 250 | mg |
| Green Tea (8% caffeine) | 100 | mg |
| Magnesium Stearate | 5 | mg |
| Silicon Dioxide | 2 | mg |

| | |
|---|---|
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the tryptophan, green tea and the other excipients. The ingredients are accurately dosed into a hard gelatin capsule. One or two capsules may be taken daily during winter or other times when the subject is expected to have low Vitamin D levels to increase energy, relieve stress or enhance overall mood.

### Example No. 29

### A Capsule Containing 5000 IU Cholecalciferol, Allium Sativum and Eleutherococcus Senticosus

| Ingredient | Amount per Capsule | |
|---|---|---|
| Vitamin D3 | 125 | μg |
| (cholecalciferol) | | |
| Allium sativum | 180 | mg |
| Eleutherococcus | 64 | mg |
| senticosus extract | | |
| Magnesium Stearate | 5 | mg |
| Silicon Dioxide | 2 | mg |

| | |
|---|---|
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the allium sativum, eleutherococcus senticosus extract and the other excipients. The ingredients are accurately dosed into a hard gelatin capsule. One or two capsules may be taken daily during winter or other times when the subject is expected to have low Vitamin D levels to increase energy, relieve stress or enhance overall mood.

### Example No. 30

### A Capsule Containing 5000 IU Cholecalciferol, 325 mg Acetaminophen, 25 mg Dextromethorphan, 30 mg Pseudoephedrine and Green Tea

| Ingredient | Amount per Capsule | |
|---|---|---|
| Vitamin D3 | 125 | μg |
| (cholecalciferol) | | |
| Acetaminophen | 325 | mg |
| Dextromethorphan | 25 | mg |
| HBr | | |
| Pseudoephedrine HCl | 30 | mg |
| Green Tea (10% caffeine) | 300 | mg |
| Magnesium Stearate | 5 | mg |
| Silicon Dioxide | 2 | mg |

| | |
|---|---|
| Microcrystalline | q.s. to Volume |
| Cellulose | |

The Vitamin D3 may be diluted serially in the cellulose, or granulated with the cellulose or spray-dried onto the cellulose. The diluted Vitamin D3 is then mixed with the acetaminophen, dextromethorphan, pseudoephedrine, green tea and the other excipients. The ingredients are accurately dosed into a hard gelatin capsule. One or two capsules may be taken daily during winter or other times when the subject is expected to have low Vitamin D levels to increase energy, relieve stress or enhance overall mood.

### Example No. 31

### A Liquid Composition Containing 2000 IU Cholecalciferol Per Dose

| Ingredient | Amount | |
|---|---|---|
| Vitamin D3 | 333 | μg |
| (cholecalciferol) | | |
| Green Tea (10% caffeine) | 3.5 | mg |
| Ethanol (95%) | 33.3 | mg |
| Preservative | 0.1 | g |
| Flavor | 0.1 | g |
| Color | 0.01 | g |

| | |
|---|---|
| Water | QS to 100 ml |

The Vitamin D3 may be dissolved in the ethanol. The Vitamin D3 solution is then mixed with the green tea and other excipients. A dose would be 15 ml. One or two doses may be taken daily during winter or other times when the subject is expected to have low Vitamin D levels to increase energy, relieve stress or enhance overall mood.

### Example No. 32

### A Solid Composition Containing 1000 IU Cholecalciferol Per Dose

| Ingredient | Amount | |
|---|---|---|
| Thiamine (B1) | 2.8 | mg |
| Riboflavin (B2) | 3.2 | mg |
| Niacin (B3) | 5 | mg |
| Pantothenic Acid (B5) | 6 | mg |
| Pyridoxine (B6) | 5 | mg |
| Biotin (B7) | 160 | μg |
| Folic Acid (B9) | 400 | μg |
| Cobalamin (B12) | 3 | μg |
| Ascorbic Acid (C) | 300 | mg |
| Cholecalciferol (D3) | 12.5 | μg |
| Green Tea | 5.00% | |
| (10% caffeine) | | |
| Sucrose | 200 | mg |

| | |
|---|---|
| Flavor | QS |
| Color | QS |

The Vitamins may be premixed and granulated with the sucrose. The powder may be packaged in sachets. A dose may be two sachets. One or two sachets may be taken daily during winter or other times when the subject is expected to have low Vitamin D levels to increase energy, relieve stress or enhance overall mood.

### Example No. 33

### A Solid Composition Containing 1000 IU Cholecalciferol Per Dose

| Ingredient | Amount | |
|---|---|---|
| Thiamine (B1) | 2.8 | mg |
| Riboflavin (B2) | 3.2 | mg |
| Pyridoxine (B6) | 3.23 | mg |
| Cobalamin (B12) | 3 | μg |
| Ascorbic Acid (C) | 300 | mg |
| Cholecalciferol (D3) | 12.5 | μg |
| Eleutherococcus | 64 | mg |
| senticosus | | |
| Sucrose | 500 | mg |

| | |
|---|---|
| Flavor | QS |
| Color | QS |

The Vitamins may be premixed and granulated with the sucrose. The powder may be packaged in sachets. A dose may be two sachets. One or two sachets may be taken daily during winter or other times when the subject is expected to have low Vitamin D levels to increase energy, relieve stress or enhance overall mood.

### Example No. 34

### A Capsule Containing 5000 IU Cholecalciferol, Rhodiola Rosea and Eleutherococcus Senticosus

| Ingredient | Amount per Capsule | |
|---|---|---|
| Vitamin D3 | 125 | μg |
| (cholecalciferol) | | |
| Rhodiola rosea | 360 | mg |
| Eleutherococcus | 64 | mg |
| senticosus extract | | |
| Magnesium Stearate | 5 | mg |
| Silicon Dioxide | 2 | mg |

| | |
|---|---|
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the rhodiola rosea, eleutherococcus senticosus and the other excipients. The ingredients are accurately dosed into a hard gelatin capsule. One or two capsules may be taken daily during winter or other times when the subject is expected to have low Vitamin D levels to increase energy, relieve stress or enhance overall mood.

### Example No. 35

### A Capsule Containing 5000 IU Cholecalciferol, Coenzyme Q10 and Green Tea

| Ingredient | Amount per Capsule | |
|---|---|---|
| Vitamin D3 | 125 | μg |
| (cholecalciferol) | | |
| Coenzyme Q10 | 300 | mg |
| Green Tea (8% caffeine) | 100 | mg |
| Magnesium Stearate | 5 | mg |
| Silicon Dioxide | 2 | mg |

| | |
|---|---|
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the Coenzyme Q10, green tea and the other excipients. The ingredients are accurately dosed into a hard gelatin capsule. One or two capsules may be taken daily during winter or other times when the subject is expected to have low Vitamin D levels to increase energy, relieve stress or enhance overall mood.

### Example No. 36

### A Capsule Containing 5000 IU Cholecalciferol, Punica Granatum and Eleutherococcus Senticosus

| Ingredient | Amount per Capsule | |
|---|---|---|
| Vitamin D3 | 125 | μg |
| (cholecalciferol) | | |
| Punica granatum | 80 | mg |
| Eleutherococcus | 64 | mg |
| senticosus extract | | |
| Magnesium Stearate | 5 | mg |
| Silicon Dioxide | 2 | mg |

| | |
|---|---|
| Microcrystalline | q.s. to Volume |
| Cellulose | |

The Vitamin D3 may be diluted serially in the cellulose, or granulated with the cellulose, spray-dried onto the cellulose. The diluted Vitamin D3 is then mixed with the Punica granatum, Eleutherococcus senticosus extract and the other excipients. The ingredients are accurately dosed into a hard gelatin capsule. One or two capsules may be taken daily during winter or other times when the subject is expected to have low Vitamin D levels to increase energy, relieve stress or enhance overall mood.

### Example No. 37

### A Capsule Containing 2000 IU Ergocalciferol and Green Tea

| Ingredient | Amount per Capsule | |
|---|---|---|
| Vitamin D2 | 50 | μg |
| (ergocalciferol) | | |
| Green Tea (8% caffeine) | 200 | mg |
| Magnesium Stearate | 5 | mg |
| Silicon Dioxide | 2 | mg |

| | |
|---|---|
| Microcrystalline | q.s. to Volume |
| Cellulose | |

The Vitamin D2 may be diluted serially in the cellulose, or granulated with the cellulose, spray-dried onto the cellulose. The diluted Vitamin D2 is then mixed with the green tea and other excipients. The ingredients are accurately dosed into a hard gelatin capsule. One or two capsules may be taken daily during winter or other times when the subject is expected to have low Vitamin D levels to increase energy, relieve stress or enhance overall mood.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for providing energy, relieving stress, and mood enhancing benefits to a human comprising: the steps of administering to a human a composition comprising;
(a) cholecalciferol; and
(b) a tea extract.

2. The method of claim 1, wherein said composition comprising from about 450 to about 500,000 IU of cholecalciferol, per dose of composition.

3. The method of claim 1, wherein said composition comprising from about 500 to about 500,000 IU of cholecalciferol, per dose of composition.

4. The method of claim 1, wherein said composition comprising from about 1000 to 100,000 IU of cholecalciferol, per dose of composition.

5. The method of claim 1, wherein said composition comprising from about 10,000 to 50,000 IU of cholecalciferol, per dose of composition.

6. The method of claim 1, wherein said human having an increase in a blood level of 25-hydroxycholecalciferol of from about 1 ng/ml to about 40 ng/ml in said human.

7. The method of claim 1, wherein said human having an increase in a blood level of 25-hydroxycholecalciferol of from about 2 ng/ml to about 30 ng/ml.

8. The method of claim 1, wherein said human having an increase in a blood level of 25-hydroxycholecalciferol of from about 4 ng/ml to about 20 ng/ml.

9. The method of claim 1, said composition further at least one additional component selected from the group consisting of a tea extract, Vitamin A, probiotics, Vitamin C, andrographis extract, 1-tryptophan, Allium sativum, Eleutherococcus senticosus, Rhodiola rosea, Coenzyme Q10, superfruits extract, and combinations thereof.

10. The method of claim 1, said composition further comprising a respiratory ingredient selected from the group consisting of analgesics, anticholinergics, antihistamines, anti-inflammatories, antipyretics, antitussives, antivirals, decongestants, expectorants, mucolytics, and combinations thereof.

11. The method of claim 1, wherein said composition is an oral composition.

12. The method of claim 11, wherein said oral composition is selected from the group consisting of liquid compositions, nasal compositions, beverage, supplemental water, pills, tablets, capsules, gel compositions, sachets and combinations thereof.

13. The method of claim 11, wherein said oral composition is said nasal composition.

14. The method of claim 12, wherein said nasal composition comprises calcitriol.

15. The method of claim 13, wherein said nasal composition is intranasally delivered to the respiratory tract of said human.

16. The method of claim 1, wherein said composition is administered as a dose as-needed, as-desired, on a less than daily dosing, daily dosing, multiple times daily, and combinations thereof.

17. A method for providing energy, relieving stress, and mood enhancing benefits to a human comprising: the steps of administering to a human a composition comprising;
(a) cholecalciferol;
(b) a tea extract; and
(b) a probiotic.

18. A method for providing energy, relieving stress, and mood enhancing benefits to a human comprising: the steps of administering to a human a composition comprising;
(a) cholecalciferol; and
(b) at least one additional component selected from the group consisting of a tea extract, Vitamin A, probiotics, Vitamin C, andrographis extract, l-tryptophan, Allium sativum, Eleutherococcus senticosus, Rhodiola rosea, Coenzyme Q10, superfruits extract and combinations thereof.

19. An oral composition for providing energy, relieving stress, and mood enhancing benefits to a human comprising: the steps of administering to a human a composition comprising, from about 450 IU to about 500,000 IU of cholecalciferol, per dose of composition; and a tea extract.

20. The composition of claim 19, further comprising at least one additional component selected from the group consisting of a tea extract, Vitamin A, probiotics, Vitamin C, andrographis extract, l-tryptophan, Allium sativum, Eleutherococcus senticosus, Rhodiola rosea, Coenzyme Q10, superfruits extract, and combinations thereof.

21. The composition of claim 19, further comprising Vitamin D₂.

22. The composition of claim 21, further comprising a respiratory ingredient selected from the group consisting of analgesics, anticholinergics, antihistamines, anti-inflammatories, antipyretics, antitussives, antivirals, decongestants, expectorants, mucolytics, and combinations thereof.

23. The composition of claim 19, wherein said composition is an oral composition.

24. The composition of claim 23, wherein said oral composition is selected from the group consisting of liquid compositions, nasal compositions, beverage, supplemental water, pills, tablets, capsules, gel compositions, sachets and combinations thereof.

25. The composition of claim 24, wherein said oral composition is said nasal composition.

26. The composition of claim 25, wherein said nasal composition comprises calcitriol.

27. The composition of claim 19, wherein said composition further comprising a respiratory ingredient selected from the group consisting of analgesics, anticholinergics, antihistamines, anti-inflammatories, antipyretics, antitussives, antivirals, decongestants, expectorants, mucolytics, and combinations thereof.

28. The composition of claim 19, wherein said composition is administered as a dose as-needed, as-desired, on a less than daily dosing, daily dosing, multiple times daily, and combinations thereof.

29. A method of providing energy, relieving stress, and mood enhancing benefits to a human comprising: the steps of administering to a human a composition comprising from about 450 to about 500,000 IU of Vitamin D₂, per dose of composition; and tea extract.

30. The method of claim 28, wherein said human having an increase in a blood level of 25-hydroxycholecalciferol of from about 1 ng/ml to about 40 ng/ml in said human.

31. The method of claim 29, said composition further at least one additional component selected from the group consisting of a tea extract, Vitamin A, probiotics, Vitamin C, andrographis extract, l-tryptophan, Allium sativum, Eleutherococcus senticosus, Rhodiola rosea, Coenzyme Q10, superfruits extract, and combinations thereof.

32. The method of claim 29, said composition further comprising a respiratory ingredient selected from the group consisting of analgesics, anticholinergics, antihistamines, anti-inflammatories, antipyretics, antitussives, antivirals, decongestants, expectorants, mucolytics, and combinations thereof.

33. The method of claim 29, wherein said composition is an oral composition.

34. The method of claim 29, wherein said composition is administered as a dose as-needed, as-desired, on a less than daily dosing, daily dosing, multiple times daily, and combinations thereof.

35. A method of providing energy, relieving stress, and mood enhancing benefits to a human comprising,
(a) cholecalciferol;
(b) Vitamin D₂; and
(c) tea extract.

36. The method of claim 34, said composition further at least one additional component selected from the group consisting of a tea extract, Vitamin A, probiotics, Vitamin C, andrographis extract, l-tryptophan, Allium sativum, Eleutherococcus senticosus, Rhodiola rosea, Coenzyme Q10, superfruits extract, and combinations thereof.

37. The method of claim 34, wherein said composition further comprising a respiratory ingredient selected from the group consisting of analgesics, anticholinergics, antihistamines, anti-inflammatories, antipyretics, antitussives, antivirals, decongestants, expectorants, mucolytics, and combinations thereof.

38. The method of claim 34, wherein said composition is administered as a dose as-needed, as-desired, on a less than daily dosing, daily dosing, multiple times daily, and combinations thereof.

39. A method for providing energy, relieving stress, and mood enhancing benefits to a human comprising: the steps of administering to a human a composition comprising;
(a) cholecalciferol; and
(b) Eleutherococcus senticosus.

40. The method of claim 39, wherein said composition comprising from about 450 to about 500,000 IU of cholecalciferol, per dose of composition.

41. The method of claim 39, wherein said human having an increase in a blood level of 25-hydroxycholecalciferol of from about 1 ng/ml to about 40 ng/ml in said human.

42. The method of claim 39, said composition further at least one additional component selected from the group consisting of a tea extract, Vitamin A, probiotics, Vitamin C, andrographis extract, l-tryptophan, Allium sativum, Eleutherococcus senticosus, Rhodiola rosea, Coenzyme Q10, superfruits extract, and combinations thereof.

43. The method of claim 39, wherein said composition is an oral composition.

44. The method of claim 44, wherein said oral composition is selected from the group consisting of liquid compositions, nasal compositions, beverage, supplemental water, pills, tablets, capsules, gel compositions, sachets and combinations thereof.

45. The method of claim 39, wherein said composition is administered as a dose as-needed, as-desired, on a less than daily dosing, daily dosing, multiple times daily, and combinations thereof.
